(19) United States
(12) Patent Application Publication
(10) Pub. No.: US 2026/0157661 A1
(43) Pub. Date: Jun. 11, 2026

(54) METHOD FOR INTERVAL THRESHOLD AUDIOMETRY

(71) Applicant: HEARING REHABILITATION CENTER "MELFON", LTD., Moscow (RU)

(72) Inventors: Valeriy Stepanovich SIROTKIN, g Moskva (RU); Vladimir Vladimirovich KHANYKOV, g Moskva (RU); Sergey Mikhailovich PASYUTA, g. Moskva (RU)

(73) Assignee: HEARING REHABILITATION CENTER "MELFON", LTD., Moscow (RU)

(21) Appl. No.: 19/327,894

(22) Filed: Sep. 12, 2025

(30) Foreign Application Priority Data
Dec. 5, 2024 (RU) 2024136497

(51) Int. Cl. A61B 5/12; A61B 5/00

(52) U.S. Cl. CPC A61B 5/123; A61B 5/742

(57) ABSTRACT

The invention relates to medicine, namely to surdology-otorhinolaryngology, and can be used in audiometry for diagnostic purposes during mass preventive examinations of the population and establishing the hearing threshold. Interval threshold audiometry is performed step by step at the following frequencies: 1000, 1500, 2000, 3000, 4000, 6000, 8000, 10000, 12500, 12500, 14000, 16000, 750, 500, 250, 125 Hz. At the same time the volume level of the starting signal is set taking into account the dependence of thresholds of perception of tone signals on the age of the test subject according to Table 1 and present the signal for all frequencies in the above-mentioned order with receiving a response from the examinee “There is a signal” or “There is no signal” at each frequency. For each frequency tested:

If a “No Signal” response is received and no “Signal is” response was previously received on this frequency, increase the signal level by 6 dB and present the signal with a “Signal is” or “No Signal” response from the subject;

If a “Signal is present” response is received and no “No Signal” response has been received on this frequency previously, reduce the signal level by 6 dB and present the signal with a “Signal is present” or “No Signal” response from the subject;

If the signal level is greater than or equal to 90 dB or less than or equal to 6 dB, the test at the selected frequency is completed;

If the answer “No signal” is received, and earlier the answer “Signal is” was received, or the answer “No signal” is received, and earlier the answer “Signal is” was received, then fix the interval with the range of levels in 6 dB, find the average value of volume levels at the same frequency at which the last answers were received: “No signal”, and after increasing or decreasing the volume level by 6 dB-“Signal is”, performs an additional test with the average volume level of the signal from the found interval, fixes the test result at the selected frequency with an accuracy of +1.5 dB and completes the test at the selected frequency. The method allows determining the upper limit of sound perception thresholds individually for each patient and, if hearing correction is necessary, selecting the optimal treatment and/or further examination.

### TECHNICAL FIELD

The present invention relates to medicine, namely to surdology-otorhinolaryngology, and can be used in audiometry for diagnostic purposes during mass preventive examinations of the population and establishing the hearing threshold.

### BACKGROUND

Tone hearing screening is up to now practically the only way to get “at least some” instrumental assessment of auditory perception in systematically conducted mass examinations in schools. Successful mastering of educational material to a large extent determines not only the successful future of the student, but also the level of future scientific and technological development of the society in which today's students will work. Numerous studies have proven that even a slight hearing impairment significantly reduces the ability to learn. WHO has developed a criterion for “educationally significant hearing loss”-ESHL “Educationally Significant Hearing Loss” (AAA, 2011; ASHA, 1997).

ESHL is defined as hearing loss that interferes with a student's academic performance (WHO, 2014). This can include permanent sensorineural, conductive and mixed hearing loss, as well as transient conductive loss. However, the severity of hearing loss that constitutes ESHL is not always clearly defined. According to the World Health Organization (2014), a disabling hearing loss in children is an average hearing threshold in the better ear at 0.5, 1, 2, 4 kHz of >30 dB HL.”

In order to reduce labor costs, all existing methods use a narrowed to the limit range of investigated “speech” frequencies: 500, 1000, 2000, 4000 Hz, although it is a well-known fact that high speech frequencies-over 6000 Hz—have a great influence on speech intelligibility.

The disadvantages of this development is a single test sequence at some given test signal level. All frequencies declared for examination are scanned sequentially. This is a traditional method of screening tonal audiometry performed on an audiometer with manual setting of the test signal volume level. When examining a group of patients, the desired test signal level is set and the entire group is examined at that value. If it is necessary to change the level, the procedure is repeated. The patient is tested at only one test signal level per pass.

The use of manually operated audiometers leads to significant time costs, resulting in the need to use only one volume level of the scanning test signal. The prognostic efficiency of the method is drastically reduced and the burden on the health care system is multiplied by the large number of unconfirmed referrals for additional testing.

From the prior art, a method of two-stage examination of hearing organs in preschool children. At the first stage, the method of tympanometry is used, and at the second stage, the method of otoacoustic emission at the distortion product frequency is used. The examination starts with the use of the tympanometry method at the probing tone frequency of 226 Hz with the rate of pressure change from +200 to −400 daPa/s. On the basis of the obtained tympanograms, which have a peak dependence of static compliance on the change of positive or negative air pressure in the external auditory canal, the pressure in daPa/s at which the peak of compliance is registered is estimated. If the pressure in the tympanic cavity and external ear canal equalizes and the pressure at which the compliance peak is recorded is between +50 and −100 daPa/s, middle ear function is assessed as normal. Otoacoustic emission examination is continued. If middle ear pathology is detected according to the results of tympanometry, the child is referred for treatment of middle ear pathology. Then after 3 weeks, tympanometry is repeated and the examination by the method of otoacoustic emission at the frequency of the distortion product is continued. The registration criterion is the ratio of the emission power to the background noise power +6 dB in 70% of frequency bands. If the results of otoacoustic emission at the frequency of the distortion product reveal pathology, the child is referred for examination to a surdological center. The method allows to increase reliability and objectivity of control of pathology of the auditory apparatus in children of preschool age (RU 2759485 C1 15.11.2021).

A method and a hardware-software complex for performing diagnostic procedures in terms of performing a pre-hospital preliminary classification multifactor assessment of the human auditory analyzer capability during mass preventive examinations of the population is known from the prior art. A method is proposed for pre-medical preliminary classifying multifactorial assessment of the capabilities of a human auditory analyzer during mass preventive examinations of the population, performed using a computing device connected to audio playback devices and containing stages in which: using a computing device, a primary test speech sequence (TSS) is formed, which consists of sentences consisting of the first number of words based on the matrix test; generate a competing noise sound for the primary TSS; the primary TSS is reproduced using audio signal reproduction devices made in the form of air and bone sound transmission headphones, while the TSS is reproduced simultaneously with the noise competing sound at the first signal-to-noise ratio using speech simulation based on a deep machine learning model; the user's oral response is received; the user's oral response is automatically analyzed by recognizing the TSS converting it to text format and analyzing the correctness of the answer using a machine learning model; moreover, based on the analysis of the user's oral responses, a dynamic change in the complexity of the assessment is carried out, in which, as a result of each automatic analysis, the number of words in sentences forming the TSS and/or the signal-to-noise ratio of the reproduced signal is changed; the user's auditory analyzer is evaluated based on the responses during playback of the test speech sequence. The invention provides an automated pre-hospital preliminary classification assessment of the capability of the human auditory analyzer during mass preventive examinations of the population (RU 2765108 C1 25.01.2022).

In addition, a method is known from the state of the art, in which screening audiometry is carried out by means of semi-automatically feeding a sequence of tone signals of a standard set of frequencies separately into each of the channels of the playback device with fixation of the user's response to recognize the tone signals. Playback of a test sequence of monotone signals in an extended frequency range for construction of a screening audiogram (RU 2743049 C1 15.02.2021).

The disadvantages of this development is a single test sequence at some given level of the test signal. All frequencies reported for examination are scanned sequentially. This is a traditional method of screening tonal audiometry performed on an audiometer with manual setting of the test signal volume level. When examining a group of patients, the desired test signal level is set and the entire group is examined at that value. If it is necessary to change the level, the procedure is repeated. The patient is tested at only one test signal level per pass.

### SUMMARY

The claimed invention solves a technical problem of the existing generally accepted method of performing tone-threshold audiometry-disproportionately long test performance time, often exceeding 12-15 minutes of testing of one ear. Children and elderly patients are physically unable to maintain the necessary concentration of attention to detect the test signal at extremely low values in the entire frequency range, which led to the need for an extreme reduction in the frequency range (500, 1000, 2000, 4000 Hz) to the detriment of information and diagnostic value. The claimed method provides reduction of testing time by 3-5 times, while providing diagnostics in the range up to 8000 Hz in the presence of hearing impairment.

The technical result is to provide the possibility of rapid assessment of hearing, in a short period of time during mass preventive examinations of the population, to increase the accuracy of determining the hearing threshold, which allows to detect hearing loss.

The following techniques are used to reduce the examination time:
1. The initial signal level on each frequency is selected based on the patient's age and the result of tests on neighboring frequencies;
2. Instead of the standard multi-step procedure of searching for a specific value of the listening threshold at a given frequency, it is proposed to determine only an interval of values within which the desired value of the listening threshold at a given frequency is guaranteed to be located. By setting an acceptable value of the error in determining the listening threshold below the standard uncertainty level (GOST R ISO 8253-1-2012/ISO 8253-1:2010), it is possible to significantly reduce the testing time without reducing the reliability of the results.

It is proposed to use the value of the acceptable uncertainty zone of +/−1.5 dB, which gives the value of the target interval width of finding the “true” value of the desired threshold of 3 dB.

The value of the standard uncertainty of measurement of the threshold level of listening in accordance with GOST R ISO 8253-1-2012/ISO 8253-1:2010: 2.5 dB at frequencies up to 4 kHz and 4 dB at frequencies above 4 kHz for measurements on air conduction; 3 dB at frequencies up to 4 kHz and 5 dB at frequencies above 4 kHz for measurements on bone conduction.

The increased level step up to 6 dB allows you to quickly find the level interval at which the hearing threshold is located and then reduce this interval to 3 dB.

The claimed technical result is achieved by the following techniques.

Interval threshold audiometry is performed stepwise at the following frequencies: 1000, 1500, 2000, 3000, 4000, 6000, 8000, 10000, 12500, 14000, 16000, 750, 500, 250, 125 Hz.

At the same time set the volume level of the starting signal taking into account the dependence of thresholds of perception of tone signals on the age of the examinee according to Table 1 and present the signal on all frequencies in the above-mentioned order with receiving a response from the examinee “There is a signal” or “No signal” on each frequency.

For each frequency tested:

If a “No Signal” response is received and no “Signal is” response was previously received on this frequency, increase the signal level by 6 dB and present the signal with a “Signal is” or “No Signal” response from the subject.

If a “Signal is present” response is received and no “No Signal” response has been received on this frequency previously, reduce the signal level by 6 dB and present the signal with a “Signal is present” or “No Signal” response from the subject.

If the signal level is greater than or equal to 90 dB or less than or equal to 6 dB, the test at the selected frequency is completed.

If the answer “No signal” is received and earlier the answer “Signal is” was received or the answer “No signal” is received and earlier the answer “Signal is” was received, then fix the interval with the range of levels in 6 dB, find the average value of volume levels on the same frequency on which the last answers were received: “No signal”, and after increasing or decreasing the volume level by 6 dB-“Signal is”, performs an additional test with the average volume level of the signal, fixes the test result at the selected frequency with an accuracy of +1.5 dB and completes the test.

The claimed method can be realized by means of air or bone conduction headphones.

TABLE 1

| | | No | | | | | | | | | | | | | | |
|---|---|---|---|---|---|---|---|---|---|---|---|---|---|---|---|---|
| | | 0 | 1 | 2 | 3 | 4 | 5 | 6 | 7 | 8 | 9 | 10 | 11 | 12 | 13 | 14 |
| Age | | Frequency (Hz) | | | | | | | | | | | | | | |
| (years) | | 125 | 250 | 500 | 750 | 1K | 1.5K | 2K | 3K | 4K | 6K | 8K | 10K | 12.5K | 14K | 16K |
| 0-35 | Test signal | 19 | 18 | 17 | 17 | 17 | 17 | 18 | 19 | 20 | 22 | 23 | 25 | 27 | 29 | 31 |
| 36-69 | level (dB) | 26 | 23 | 20 | 20 | 20 | 21 | 22 | 25 | 27 | 30 | 33 | 37 | 41 | 44 | 48 |
| 70+ | | 30 | 28 | 25 | 24 | 24 | 25 | 27 | 30 | 33 | 38 | 43 | 48 | 54 | 59 | 65 |

### BRIEF DESCRIPTION OF DRAWINGS

FIG. 1 illustrates the test protocols of Example #1.

FIG. 2 illustrates the test protocols of Example #2.

FIG. 3 illustrates the test protocols of Example #3.

FIG. 4 illustrates the test protocols of Example #4.

### DETAILED DESCRIPTION OF THE INVENTION

In order to eliminate the existing shortcomings in the determination of the hearing threshold level for hearing assessment, in order to organize multilevel screening and examination, for example, in schools, it is possible to recommend the claimed method with consecutive testing of one patient with the use of individually selected starting volume level of the test signal, providing operative possibility to determine the hearing threshold and the level of perception of tone signals of each individual. The present method allows to reduce significantly the time of the actual process of testing of one patient when carrying out the stated procedure in the prescribed order of execution of the present algorithm in mass examination, for example, in school conditions.

TABLE 2
The internationally recognized hearing loss scale.

| | Hearing thresholds, | Degree of severity |
|---|---|---|
| Degree of violation | dB | violations |
| normal | “−10”-25 | normal |
| Grade I hearing loss | 26-40 | light |
| Grade II hearing loss | 41-55 | average |
| Grade III hearing loss | 56-70 | medium |
| Grade IV hearing loss | 71-90 | heavy |
| Deafness | More than 91 | deafness |

So the method of interval threshold audiometry developed by us is carried out as follows: the subject is gradually presented with a tone signal at the following frequencies: 1000, 1500, 2000, 3000, 4000, 6000, 8000, 10000, 12500, 14000, 16000, 750, 500, 250, 125 Hz. In this case, the volume level of the starting signal is set taking into account the dependence of thresholds of perception of tone signals on the age of the test subject according to Tab. 1 and present the signal on all frequencies in the above-mentioned order with receiving a response from the examinee “There is a signal” or “There is no signal” on each frequency.

For each frequency tested:

If a “No Signal” response is received and no “Signal is” response was previously received on this frequency, increase the signal level by 6 dB and present the signal with a “Signal is” or “No Signal” response from the subject.

If a “Signal is present” response is received and no “No Signal” response has been received on this frequency previously, reduce the signal level by 6 dB and present the signal with a “Signal is present” or “No Signal” response from the subject.

If the signal level is greater than or equal to 90 dB or less than or equal to 6 dB, the test at the selected frequency is completed, considering the specified signal levels as threshold.

If the answer “No signal” is received and earlier the answer “Signal is” was received or the answer “No signal” is received and earlier the answer “Signal is” was received, then fix the interval with the range of levels in 6 dB, find the average value of volume levels on the same frequency on which the last answers were received: “No signal”, and after increasing or decreasing the volume level by 6 dB-“Signal is”, performs an additional test with the average signal volume level, fixes the test result at the selected frequency with an accuracy of +1.5 dB and completes the test, considering the obtained signal level as threshold.

Thus, the scheme for determining the level of hearing threshold starting from the starting volume level provides a significant reduction in the total scanning time and makes it possible to detect hearing impairments that can only be picked up using the specified extended frequency range.

The claimed method can be realized on the basis by means of hardware-software complex Melfon (RU 2743049 C1 15.02.20).

The claimed method can be realized by means of air or bone conduction headphones.

In this way, the upper limit of hearing thresholds is determined for each individual. This indicator is critical in developing a correction scheme for the degree of hearing loss identified.

### Examples

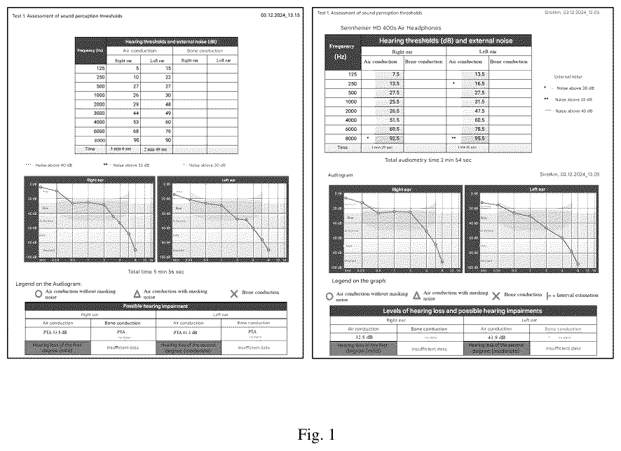

1. An example of a 6 dB interval audiogram is shown in the full test report (FIG. 1) compared to a “classic” audiogram. Both audiograms were taken with minimal time delay. It becomes possible for the first time to assess the key indicator of hearing loss severity—the average threshold value at 4 basic frequencies 500-1000-2000-4000 Hz-already at the screening stage.
The PTA (Pure Tone Average) values from the full test protocol are shown in Table 3. The results obtained by different methods are almost identical.

TABLE 3
PTA performance based on test results

| Ear | Algorithm | PTA, dB | Diagnosis |
|---|---|---|---|
| Right | Interval | 32.8 | Hearing loss |
| | Classic | 33.8 | first degree (lung) |
| Left | Interval | 41.3 | Hearing loss |
| | Classic | 41.8 | second-degree (moderately severe) |

Table 4 summarizes the results of comparisons of test times by different methods.

TABLE 4
Time comparisons of classical and interval methods.

| Comparables | Classical | Interval | Distinction | |
|---|---|---|---|---|
| parameters | audiometry | audiometry | results | Assessment |
| Right ear. | 186 | 89 | +97 sec. | Reduced |
| Total time (sec) | | | | 2.1 times |
| Left ear. | 169 | 85 | +84 sec. | Reduced |
| Total time (sec) | | | | twofold |

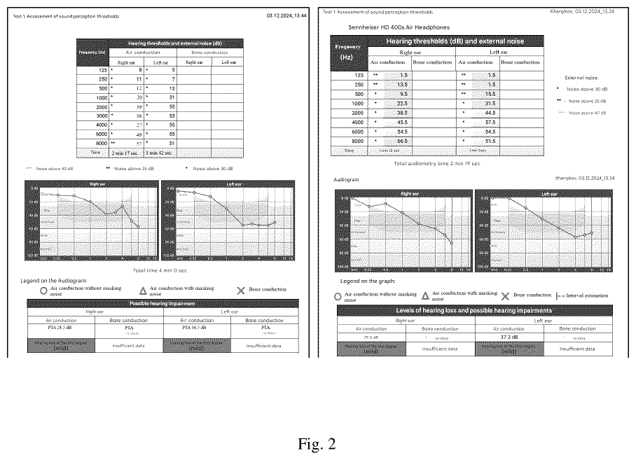

2. An example of a 6 dB interval audiogram is shown in the full test report (FIG. 2) of a hearing-impaired man born in 1946 compared to a “classic” audiogram. Both audiograms were taken with minimal time delay.

The PTA (Pure Tone Average) values from the full test protocol are summarized in Table 5. The results obtained by different methods are almost identical.

TABLE 5
PTA performance based on test results

| Ear | Algorithm | PTA, dB | Diagnosis |
|---|---|---|---|
| Right | Interval | 28.5 | Hearing loss |
| | Classic | 29 | first degree (lung) |
| Left | Interval | 37.3 | Hearing loss |
| | Classic | 38.5 | first degree (lung) |

Table 6 summarizes the results of comparisons of test times between the different methods.

TABLE 6
Comparisons of the timing of the classical and screening methods.

| Comparables | Classical | Interval | Distinction | |
|---|---|---|---|---|
| parameters | audiometry | screening | results | Assessment |
| Right ear. | 137 | 70.5 | +66.5 sec. | Reduced |
| Total time (sec) | | | | 1.9 times |
| Left ear. | 102 | 63 | +39 sec. | Reduced |
| Total time (sec) | | | | 1.6 times |

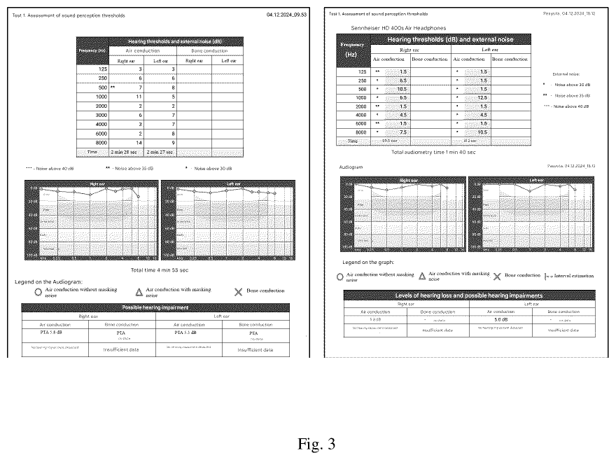

3. An example of a 6 dB interval audiogram is shown in the full test report (FIG. 3) of a male born in 1997 with hearing loss compared to a “classic” audiogram. Both audiograms were taken with minimal time delay.

The PTA (Pure Tone Average) values from the full test protocol are summarized in Table 7. The results obtained by different methods are almost identical.

TABLE 7
PTA performance based on test results

| Ear | Algorithm | PTA, dB | Diagnosis |
|---|---|---|---|
| Right | Interval | 5.8 | No hearing impairment was |
| | Classic | 5.8 | detected. |
| Left | Interval | 5.0 | No hearing impairment was |
| | Classic | 5.5 | detected. |

Table 8 summarizes the results of comparisons of test times between the different methods.

TABLE 8
Time comparisons between classical and screening methods

| Comparables | Classical | Interval | Distinction | |
|---|---|---|---|---|
| parameters | audiometry | screening | results | Assessment |
| Right ear. | 148 | 41.2 | +106.8 sec. | Reduced |
| Total time (sec) | | | | 3.6 times |
| Left ear. | 147 | 59.5 | +87.5 sec. | Reduced |
| Total time (sec) | | | | 2.5 times |

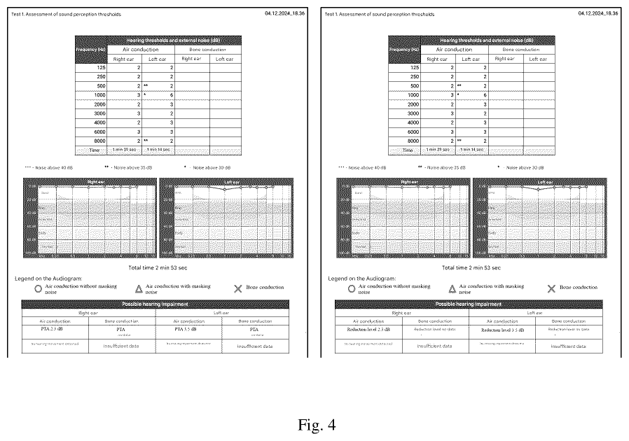

4. An example of a 6 dB interval audiogram is shown in the full test report (FIG. 4) of a hearing-impaired woman born in 1995 compared to a “classic” audiogram. Both audiograms were taken with minimal time delay.

The PTA (Pure Tone Average) values from the full test protocol are summarized in Table 9. The results obtained by different methods are almost identical.

TABLE 9
PTA performance based on test results

| Ear | Algorithm | PTA, dB | Diagnosis |
|---|---|---|---|
| Right | Interval | 1.5 | No hearing impairment was |
| | Classic | 2.3 | detected. |
| Left | Interval | 2.8 | No hearing impairment was |
| | Classic | 3.5 | detected. |

Table 10 summarizes the results of comparisons of test times between the different methods.

TABLE 10
Time comparisons of classical and screening methods.

| Comparables | Classical | Interval | Distinction | |
|---|---|---|---|---|
| parameters | audiometry | screening | results | Assessment |
| Right ear. | 99 | 36 | +63 sec. | Reduced |
| Total time (sec) | | | | 2.8 times |
| Left ear. | 74 | 31.2 | +42.8 sec. | Reduced |
| Total time (sec) | | | | 2.4 times |

1. A method of performing interval threshold audiometry performed in stages at the following frequencies: 1000, 1500, 2000, 3000, 4000, 6000, 8000, 10000, 12500, 12500, 14000, 16000, 750, 500, 250, 125 Hz; wherein
set the volume level of the starting signal taking into account the dependence of thresholds of perception of tone signals on the age of the examinee according to Table 1 and present the signal on all frequencies in the above-mentioned order with receiving a response from the examinee “There is a signal” or “No signal” on each frequency;
for each frequency under test:
if a “No Signal” response is received and no “Signal is” response was received earlier on this frequency, increases the signal level by 6 dB and presents the signal with a “Signal is” or “No Signal” response from the examinee;
if a “Signal is present” response is received and no “No Signal” response was received earlier on this frequency, reduces the signal level by 6 dB and presents the signal with a “Signal is present” or “No Signal” response from the examinee;
if the signal level is greater than or equal to 90 dB or less than or equal to 6 dB, the test at the selected frequency is completed;
if the answer “No signal” is received and earlier the answer “Signal is” was received or the answer “No signal” is received and earlier the answer “Signal is” was received, then fix the interval with the range of levels in 6 dB, find the average value of volume levels at the same frequency at which the last answers were received: “No signal”, and after increasing or decreasing the volume level by 6 dB-“Signal is”, performs an additional test with the average volume level of the signal, fixes the test result at the selected frequency with an accuracy of +1.5 dB and completes the test.

2. The method according to claim 1, wherein the earpieces are air or bone conduction.
